(19) United States
(12) Patent Application Publication
(10) Pub. No.: US 2006/0165623 A1
(43) Pub. Date: Jul. 27, 2006

(54) Natural deodorant composition

(75) Inventors: Tanya Workman, Mansonville (CA); Svetlana Ratnikova, Toronto (CA)

Correspondence Address: Louis C. Paul, Esq.;CTSW, Suite 2400, 420 Lexington Avenue, New York, NY, 10170, US

(73) Assignee: Terra Firma Natuals, Inc.

(21) Appl. No.: 11/042,569

(22) Filed: Jan. 24, 2005

(51) Int. Cl. A61K 8/73

(52) U.S. Cl. 424/65; 424/70.13

(57) ABSTRACT

The present invention relates to a natural deodorant system and a natural system for topical and systemic delivery of active ingredients, both systems being primarily free of, preferably substantially free of, more preferably essentially free of, and most preferably completely free of ethoxylates or other petrochemical derivatives, and comprising: (a) at least one of (1) glycerine (preferably of plant origin), (2) a polyol selected from the group consisting of galactitol, erythritol, inositol, ribitol, dithioerythritol, dithiothreitol, (3) a sugar alcohol, selected from the group consisting of mannitol, sorbitol, xylitol and maltitol, (4) a hydrogenated starch hydrosylates of at least one of berries, apples or plums, and (5) mixtures thereof; (b) water or a lower monohydric alcohol, selected from the group of methanol, ethanol, propanol and isoproponal, or mixtures thereof, present at a combined concentration of at least 20%; (c) one or more carrageenans (preferably of plant origin) or alginates, or mixtures thereof, present in combined concentrations of less than about 2%; and (d) optionally, one or more thickeners or gums selected from the group consisting of tara, guar, xanthan, Arabic, tragacanth, agar, locust bean gum, ghatti and microcrystalline celluloses.

### CROSS-REFERENCE TO RELATED APPLICATIONS

Not Applicable.

### STATEMENT REGARDING FEDERALLY-SPONSORED RESEARCH OR DEVELOPMENT

Not Applicable.

### FIELD OF THE INVENTION

The field of invention of the present invention relates to a deodorant composition for use in cosmetic, personal care. The invention particularly relates to a natural deodorant composition comprising carrageenan and/or alginates that is primarily free of ethoxylates and other petrochemical derivatives. The invention also relates to a system for topical delivery of natural ingredients comprising carrageenan and/or alginates that is primarily free of ethoxylates and other petrochemical derivatives.

### BACKGROUND OF THE INVENTION

Perspiration produced in the apocrine sweat glands of the armpits (axilla) contains proteins and fatty acids. While initially odorless, this sweat becomes malodorous when bacteria on the skin and hair metabolize the proteins and fatty acids. The problem of odoriferous sweat breakdown products is generally addressed in two ways by personal care products—antiperspirancy and deodorancy.

Antiperspirancy commonly is achieved by topically applying a composition containing a metal salt, such as an astringent aluminum or aluminum/zirconium salt. An influx of aluminum ions into secretory coil cells of human sweat glands are accompanied by an influx of water, causing the sweat gland cells to swell, squeezing the sweat ducts closed, and stopping perspiration. An undesired consequence of antiperspirancy is that by blocking the glands that produce sweat an important conduit for excretion of toxins is impeded. Additionally, some synthetic chemicals used in antiperspirants may be associated with allergic reactions, adverse health effects and/or cause discoloration of clothing.

For the above reasons, an increasing number of consumers seek personal care products that address malodor through deodorancy. Deodorants function in several ways. Some mask the odor from bacterial breakdown products; others react with the bacterial byproducts that cause odor. Still others achieve deodorancy by inhibiting bacterial growth.

Deodorant products currently on the market contain at least one, and often several, man-made, synthetic chemical ingredient. In many deodorants, a synthetic ingredient functions as a bacteriostatic agent, (e.g., triclosan, ethylhexylglycerine, phenolic derivatives, quaternary ammonium compounds). Some synthetic ingredients also serve as emulsifiers, clarifiers, solubilizers and stabilizers, many after being ethoxylated. For purposes of the present invention, the term “stabilizer” means an ingredient that prevents breakdown of an emulsion into its component parts and/or an ingredient that prevents degradation, decomposition or loss of potency of an active ingredient. An emulsifier commonly used in deodorants is Polysorbate 20, an ethoxylated sorbitan ester.

Among the synthetic ingredients commonly used in deodorants, there have been reported associations with adverse health effects, including skin irritation, allergic reactions, chemical sensitivity and, in sensitive subpopulations (e.g., children and elderly), breakdown of the skin barrier. Some synthetic ingredients used in deodorants are the subject of ongoing investigations into potential longer-term health consequences, including toxicity and carcinogenicity. These concerns have been raised, in part, because the proximity of the underarm skin to the sweat and lymph glands could lead to systemic absorption of potentially injurious chemicals.

For the reasons mentioned above, the use of glycerin and derivatives in “natural” deodorant products is on the rise, particularly in the health foods/“natural products” market. According to one study, vegetable glycerin is now used in approximately 40% of “natural” deodorant stick formulations. Other common ingredients in “natural” deodorant glycerin sticks include sodium stearate (as an emulsifier) and lichen, grapefruit seed extract and ethylhexylglycerine (as bacteriostats). Grapefruit seed extract, however, is often mistakenly assumed to be a “natural” ingredient. A study of six commercially-available grapefruit seed extracts concluded that antimicrobial activity of this ingredient was attributable to the presence of synthetic preservative agents. Benzethonium chloride was detected by thin layer chromatography in five of the six extracts tested. Three extracts were found to contain two additional synthetic preservatives—triclosan and methyl paraben. S. Sakamoto, “Analysis of components in natural food additive ‘grapefruit seed extract’ by HPLC and LC/MS,” Pharmazie 54(6): 452-6 (June 1999). Ethylhexylglycerin, sold under the tradename Sensiva SC 50 by Schülke and Mayr, likewise is not a “natural” ingredient. According to product literature, Sensiva SC 50 is a synthetic representative of 1-alkyl glycerin ether.

Clear Enzyme Deodorant (Alba Botanica) and Herbal Clear PG Free (Herbal Care Systems, Inc.) are examples of commercially available “natural” glycerin deodorant sticks containing vegetable glycerin, sodium stearate and lichen. In order to stabilize, solubilize, emulsify and/or clarify vegetable glycerine, these “natural” deodorant stick formulation contain one or more ethoxylated ingredients. The Alba Botanica and Herbal Care products include Steareth-100, the polyethylene glycol ether of stearyl alcohol, acting as a surfactant. Alcohol ethoxylates, like Steareth-100, are commonly used in “natural” deodorant sticks. Indeed, the inventors are unaware of any prior art water-based vegetable glycerine-based deodorant system that is both stable and does not contain an ethoxylate or other petrochemical derivative.

Since their introduction more than twenty years ago, volatile silicone compounds have been used to improve application of deodorant sticks and gels. They impart a non-greasy skin feel and reduce whitening. Carrageenans are used in the present invention to achieve the same benefits but in a natural system. Carrageenan provides excellent product application, helps to forms a thin non-greasy layer which retains the antibacterial actives and does not tend to whiten clothes.

The use of carrageenans in deodorant products as part of gelling systems in personal care products is known. For example, U.S. Pat. No. 6,312,702 teaches a solid cosmetic composition free of fillers comprising water and less than 20% based on total weight of the composition of a hydrophilic gelling system. (Unless otherwise indicate, % as used in this application refers to percent by weight of the total composition.) The hydrophilic gelling system taught in the '702 patent consists essentially of a combination of at least 2% kappa-carrageenan, relative to the total weight of the composition, and at least one xanthan gum, soluble cellulose derivatives and optionally an additional carrageenan.

The present invention thus meets the long-felt, but unmet need for a natural system for topical and systemic delivery of active ingredients and a natural deodorant system, both systems being primarily free of, preferably substantially free of, more preferably essentially free of, and most preferably completely free of ethoxylates or other petrochemical derivatives.

### SUMMARY OF THE INVENTION

The invention is a natural system for topical and systemic delivery of active ingredients and a natural deodorant system, both systems being primarily free of, preferably substantially free of, more preferably essentially free of, and most preferably completely free of ethoxylates or other petrochemical derivatives, the invention natural system comprising: (a) at least one of (1) glycerine (preferably of plant origin), (2) a polyol selected from the group consisting of galactitol, erythritol, inositol, ribitol, dithioerythritol, dithiothreitol, (3) a sugar alcohol, selected from the group consisting of mannitol, sorbitol, xylitol and maltitol, (4) a hydrogenated starch hydrosylates of at least one of berries, apples or plums, and (5) mixtures thereof, (b) water or a lower monohydric alcohol, selected from the group of methanol, ethanol, propanol and isoproponal, or mixtures thereof, present at a combined concentration of at least 20%; (c) one or more carrageenans (preferably of plant origin) or alginates, or mixtures thereof, present in combined concentrations of less than 2%; and (d) at least one natural deodorant ingredient. Optionally the present invention may contain one or more of the following additional ingredients: (i) essential oils; (ii) immuno-supportive ingredients, preferably a β glucan; (iii) sodium stearate and/or soapnut; (iv) one or more thickeners or gums; (v) antioxidants such as vitamins, minerals, isolated planted based antioxidants; (vi) one or more plant extracts including, but not limited to, olive leaf, calendula, astragalus, rhodeola rosea (rose root), cleavers, green tea, white tea, red tea (rooibos), black tea.

### BRIEF DESCRIPTION OF THE DRAWINGS

Not applicable.

### DETAILED DESCRIPTION OF INVENTION

The natural deodorant system of the present invention provides a stable vehicle for delivering natural deodorant ingredients, without the need for synthetic preservatives and/or synthetic emulsifiers, and comprises: (a) at least one of (1) glycerine (preferably of plant origin), (2) a polyol selected from the group consisting of galactitol, erythritol, inositol, ribitol, dithioerythritol, dithiothreitol, (3) a sugar alcohol, selected from the group consisting of mannitol, sorbitol, xylitol and maltitol, (4) a hydrogenated starch hydrosylates of at least one of berries, apples or plums, or mixtures thereof; (b) water or a lower monohydric alcohol, selected from the group of methanol, ethanol, propanol and isoproponal, or mixtures thereof, present at a combined concentration of at least 20%; (c) one or more carrageenans (preferably of plant origin) or alginates, or mixtures thereof, present in combined concentrations of less than 2%; and (d) at least one natural deodorant ingredient. The combined concentration of water and one or more of glycerine, galactitol, erythritol, inositol, ribitol, dithioerythritol, dithiothreitol, mannitol, sorbitol, xylitol, maltitol, or hydrogenated starch hydrolysates of berries, apples or plums is at least about 60%, preferably at least about 80%, more preferably at least about 90%, and most preferably at least about 95%.

For the purposes of the present application, “of plant origin” means that the material is a portion of a plant or an extract or distillate thereof. The vehicle used to obtain the extract or distillate may be selected from any suitable material known to persons of skill in the art. Preferably, the extraction vehicle is neither a petroleum product nor an alcohol. The natural deodorant system of the claimed invention is primarily free of, preferably substantially free of, more preferably essentially free of, and most preferably completely free of ethoxylates or other petrochemical derivatives. A preferred embodiment of the present invention consists of glycerine in combination with water and one or more carrageenans.

Optionally, the present invention may contain one or more of the following additional ingredients: (i) essential oils; (ii) immuno-supportive ingredients, preferably a beta glucan, delivered to the glandular and/or lymphatic systems by percutaneous absorption through the underarm skin; (iii) sodium stearate, potassium stearate, calcium stearate, soapnut (Sapindus trifoliatus), larch extract (Larch arabinogalactan) and/or rice bran extract; (iv) one or more thickeners or gums selected from the group consisting of tara, guar, xanthan, Arabic, tragacanth, agar, locust bean gum, ghatti and microcrystalline celluloses. Sodium stearate, soapnut, larch and rice bran extracts function as emulsifiers and also control hardness and texture; (v) mineral salts including, but not limited to, salts of potassium and calcium.

In a stick deodorant embodiment of the present invention, surprisingly, a low level of carrageenan, of from about 0.6% to about 2%, in combination with sodium stearate at levels of from about 3% to about 8% was found to stabilize high levels of vegetable glycerine and water in combined concentrations of from about 88% to about 95%. Preferably in a stick embodiment, carrageenan is present at levels of from about 0.8% to about 1.5%, vegetable glycerine is present from about 35% to about 55% percent, sodium stearate is present from about 4% to about 7%, and water is present from about 35% to about 55% percent. Optionally, the stick embodiment may contain leaves, powder or liquid extracts of green tea, white tea, and/or rooibos tea.

In a cream deodorant embodiment of the present invention, having a viscosity of from about 100 to about 5000 mPa-s, surprisingly, a low level of carrageenan, of from about 0.4% to about 1%, in combination with sodium stearate at levels of from about 1% to about 4.5% was found to stabilize high levels of vegetable glycerine and water in combined concentrations of from about 88% to about 98%. Preferably in cream embodiments of the present invention, carrageenan is present at levels of from about 0.4% to about 0.8%, vegetable glycerine is present from about 15% to about 30% percent, sodium stearate is present from about 0.5% to about 4%, and water is present from about 63% to about 78% percent. Optionally, the cream embodiment may contain leaves, powder or liquid extracts of green tea, white tea, and/or rooibos tea.

In a lotion deodorant embodiment of the present invention, having a viscosity of from about 1 to about 2000 mPa-s, surprisingly, a low level of carrageenan, of from about 0.1% to about 1%, in combination with sodium stearate at levels of from about 0.1% to about 4% was found to stabilize high levels of vegetable glycerine and water in combined concentrations of from about 88% to about 98%. Preferably in cream embodiments of the present invention, carrageenan is present at levels of from about 0.1% to about 0.6%, vegetable glycerine is present from about 15% to about 30% percent, sodium stearate is present from about 0.2% to about 3%, and water is present from about 63% to about 78% percent. Optionally, the lotion embodiment may contain leaves, powder or liquid extracts of green tea, white tea, and/or rooibos tea.

In a flowable gel deodorant embodiment of the present invention, having a viscosity of from about 600 to about 30000 mPa-s, carrageenan at concentrations of from about 0.6% to about 2%, is sometimes combined with, but may exclude sodium stearate in order to acquire a clear gel. When used sodium stearate is at levels of from about 0.1% to about 4.5% and, surprisingly, was found to stabilize vegetable glycerine and water in combined concentrations of from about 88% to about 98%. Preferably in these flowable gel embodiments, carrageenan is present at levels of from about 0.6% to about 1.2%, vegetable glycerine is present from about 30% to about 35% percent, if sodium stearate is used is present from about 0.5% to about 3%, and water is present from about 50% to about 55% percent. Optionally, the gel embodiment may contain leaves, powder or liquid extracts of green tea, white tea, and/or rooibos tea.

In a spray or roll-on deodorant embodiment of the present invention, having a viscosity of from about 1 to about 15 mPa-s, carrageenan at concentrations of from about 0.1% to about 0.5%, can be combined with soapnut or liquid soap at levels of from about 0.05% to about 1% and, surprisingly, was found to stabilize vegetable glycerine and water in combined concentrations of from about 88% to about 98%. Preferably in these flowable spray embodiments, carrageenan is present at levels of from about 0.05% to about 0.2%, vegetable glycerine is present from about 20% to about 30% percent, soapnut or liquid soap is present from about 0.08% to about 0.5%, and water is present from about 65% to about 75% percent. Optionally, the spray embodiment may contain leaves, powder or liquid extracts of green tea, white tea, and/or rooibos tea.

The present invention comprises one or more natural deodorant ingredients, which term shall mean a distillate or extract derived from any portion of a plant, including, without limitation, flowers, leaves, stems, berries, rinds, fruits, pulps, resins, seeds, barks, roots, tubers, rhizomes, etc. of plants, that provides deodorant activity and is primarily free of the following synthetic preservative ingredients: triclosan, phenolic derivatives, quaternary ammonium compounds, and ethoxylates. For purposes of the present invention “deodorant activity” as used in connection with a preservative ingredient shall mean capable of killing or hindering the growth of microorganisms that generate malodor or that promote the decomposition of body oils into odiferous fatty acids.

For purposes of the present invention, the term “primarily free of” in connection with a synthetic deodorant ingredient shall mean that the ingredient is used at concentration that does not provide any significant amount of bacteriostatic activity (i.e., inhibition of microbial growth). Bacteriostatic action is considered “significant” if an 80% or greater reduction in the growth of an inoculum of a relevant micro-organism is observed, relative to a control medium, without an anti-microbial agent, over a period of 16 to 24 hours at 37° C. For deodorant products, since the substrate to be treated is human skin, a relevant micro-organism is Staphylococcus epidermidis. Methods for determining whether a synthetic ingredient has a significant amount of bacteriostatic activity are described by Balows et al., “Manual of Clinical Microbiology” (5th Edition, American Society for Microbiology, Washington D.C., 1991) and in U.S. Pat. No. 6,793,914, Col. 9, lines 1-15, the disclosure of which is hereby incorporated by reference.

Where triclosan is intended to provide a significant preservative or bacteriostatic activity in deodorant products (those not of the present invention), it is typically used at a concentrations of at least about 0.3% by weight. Accordingly, a person of ordinary skill in the art would understand that a deodorant composition primarily free of triclosan would contain triclosan in concentrations of less than 0.3%. Methyl paraben is representative of a phenolic compound commonly used in personal care products to provide preservative or bacteriostatic activity. To produce a significant amount of antimicrobial activity, methyl paraben is typically used at concentrations of at least about 0.1% by weight. A person of ordinary skill in the art would understand that a deodorant composition primarily free of methyl paraben would contain such compound at concentrations of less than 0.1%. A person of ordinary skill in the art would understand a deodorant composition primarily free of other phenolic compounds in a similar manner, i.e., providing no more deodorant activity than 0.1% of methyl paraben. Primarily free of synthetically derived quaternary ammonium compounds and ethoxylates would be understood in a manner similar to those illustrated above for triclosan and methyl paraben.

Preferred natural deodorant ingredients suitable for use in the present invention include limonoid glucosides and bioflavonoids derived from the peel, seeds and pulp of citrus fruit (Citrus limon), most preferably from the pericarp and/or flavedo or albedo layers, and lichens (Usnea) or usnic acid. A preferred natural deodorant ingredient is Bio-Citrus™, a combination of limonoid glucosides and bioflavonoids that inhibits the growth of and kills E. coli, salmonella, listeria, campylobacter and staphylococcus. Bio-Citrus™ may be used, in the present invention in concentrations of from about 0.5% to about 1.00% and is commercially available from GoVitalityGo Research, Ltd. (Otterburn Park, Quebec, Canada). Another preferred natural deodorant ingredient is Lichen extract (usnea) extracted, available from Tri-K Industries (Northvale, N.J.). Lichen extract may be used in the present invention at concentrations of from about 1 to about 10 percent. Usnic acid may be used in the present invention at concentrations of up to about 0.5%. In a most preferred embodiment, the preferred natural deodorant ingredients are used in combination, where limonoid glucosides and bioflavonoids are present at concentrations of from about 0.35% to about 0.5% and Lichen extract is present at a concentration of from about 3% to about 5% by weight.

Lambda, kappa, kappa II and iota carrageenans—processed, respectively, from Chondrus crispus, Eucheuma cottonii and Eucheuma spinosum—are suitable for use in the present invention. Each is available from FMC Corporation (Philadelphia, Pa.). Carrageenans are used as stabilizers and/or thickeners in gel formulations. Lambda carrageenan is used as a stabilizer and/or thickener in non-gelling systems. In a particularly preferred embodiment of gelling systems, lambda, iota, and kappa carrageenans are used, respectively, at concentrations of about 0.3%, about 0.3% and about 0.2%.

Another aspect of the invention is a natural system for topical and systemic delivery of natural active ingredients. For purposes of the present invention a “natural active ingredient” shall mean an ingredient derived primarily from plant, animal or mineral that is essentially free of petroleum derived and petroleum modified/reacted substances. The natural topical delivery system comprises glycerine (preferably of plant origin), water and carrageenan (preferably of plant origin) and natural deodorant ingredients, in a system that is free of, preferably substantially free of, more preferably essentially free of, and most preferably completely free of ethoxylates or other petrochemical derivatives. The delivery system can take the form a spray, roll-on, flowable gel, cream, lotion, or solid stick. Varying viscosities are achieved by combining the glycerine, water and carrageenan in the following weight/weight ratios: spray or roll-on—glycerine from about 3% to about 30%, water from about 75% to about 100%, carrageenan from about 0.05% to about 2%; flowable gel—glycerine from about 5% to about 30%, water from about 55% to about 75%, carrageenan from about 0.6% to about 1.2%; cream—glycerine from about 1% to about 20%, water from about 3% to about 90%, carrageenan from about 0.4% to about 0.8%; lotion—glycerine from about 1% to about 20%, water from about 3% to about 90%, carrageenan from about 0.1% to about 0.8%; solid stick—glycerine from about 15% to about 55%, water from about 40% to about 80%, carrageenan from about 0.06% to about 2%. The solid stick, cream and lotion embodiments may optionally contain sodium stearate, respectively at the following weight/weight concentrations: solid stick—from about 2% to about 7%; cream—from about 0.5% to about 4%; lotion—from about 0.01% to about 2%.

The natural delivery system may be used for the topical or transdermal delivery of active ingredients which are well-known to cosmetic and pharmaceutical chemists. Compositions and methods for transdermal delivery are well-known in the dermatologic, cosmetic and pharmaceutical arts and are described in U.S. Pat. No. 6,267,984, the disclosure of which is incorporated herein by reference. Examples of such active ingredients include, but are not limited to, antioxidants, anti-wrinkle agents, vitamins, sunscreen actives, moisturizers, penetration enhancers and immuno-supportive ingredients, as well as natural cosmetic ingredients, such as pigments, colorants and fragrances of plant origin as well as colorants and pigments derived from inorganic minerals. Penetration enhancers are ingredients that enhance the ability of the cell membrane to both absorb and retain pharmacologically active agents. Sunscreen actives are organic or inorganic agents that absorb or reflect light waves over a specified spectrum, thus preventing potentially harmf uil erythemal ultraviolet radiation from reaching and damaging the skin.

For purposes of the present invention, an immuno-supportive ingredient shall mean an ingredient that leads to one or more of the following physiological responses by immune cells (e.g., monocytes, macrophages, neutrophils and natural killer cells) to foreign bodies: increased production of white blood cells; mobilization of immune cells to the site of foreign invasion; enhanced phagocytosis or killing of foreign bodies; increased intracellular communication among immune cells with respect to foreign invasion; enhanced acquired immune response (i.e., by antibodies and T-cells). A preferred immuno-supportive ingredient suitable for delivery in the present invention to the glandular and lymphatic systems via percutaneous absorption through the underarm skin are 1,3 β glucans. A particularly preferred 1,3 β glucan has 1,3/1,6 glucose linkages, and is sold under the tradename WGP-6 by Biopolymer Engineering, Inc. (Eagan, Minn.).

Essential oils from the group consisting of florals, herbs, fruits, trees, shrubs and grasses may optionally be added to the present invention. Preferred essential oils suitable for use in the present invention include, but are not limited to, rosewood, lavender, litsea cubeba, tea tree, lemon, lime, orange, petitgrain, geranium, lemongrass, palmarosa, mandarin, coriander, rose, yarrow, cypress, citronella, bergamot, pine, myrtle. Fragrance, preferably natural, and/or a hydrosol (i.e., the steam distillate of a botanical) may also optionally be added. Preferred hydrosols include lemongrass, petitgrain and rose. Essential oils alone or in combination with one or more fragrances and/or hydrosols may be present in concentrations of from about 0.1% to about 5% .

The natural deodorant and natural delivery systems of the present invention are prepared according to principles and techniques generally known to those skilled in the cosmetic and pharmaceutical arts. Carrageenan is premixed in vegetable glycerine. Water is then added and heated slowly to 80° C. Water phase ingredients (i.e., ingredients that are soluble or miscible in water), including the natural deodorant ingredient and/or other active ingredient are then added. Depending on the desired delivery form (e.g., stick), sodium stearate is then added and mixed until fully dissolved. Thereafter, heat is reduced to approximately 60° C. to 65° C., at which temperature essential oils and other ingredients known to be unstable at elevated temperatures, including for example, essential oils are added and mixed until homogenously distributed.

The following examples are further illustrative of the present invention. The components and specific ingredients are presented as being typical, and various modifications can be derived in view of the foregoing disclosure within the scope of the invention. Since numerous modifications of the present invention are possible, it is understood that within the scope of the appended claims, the invention may be practiced otherwise than as specifically described herein.

### EXAMPLE 1

### Rooibos Deodorant Stick

| Ingredient | % wt/wt |
|---|---|
| Glycerine | 35.00 |
| Sodium Stearate | 5.00 |
| Kappa Carrageenan | 0.50 |
| Iota Carrageenan | 0.50 |
| Lambda Carrageenan | 0.20 |
| Bio-citrus | 0.50 |
| Lichen | 6.00 |
| Potassium Chloride | 0.05 |
| Rooibos Tea Extract | 2.00 |
| Herbal Blend - Calendula, Cat's Claw, | 0.25 |
| Olive Leaf, Cleavers | |
| Water | 50.00 |

### EXAMPLE 2

### Green Tea Deodorant Stick

| Ingredient | % wt/wt |
|---|---|
| Glycerine | 35.00 |
| Sodium Stearate | 5.00 |
| Kappa Carrageenan | 0.30 |
| Iota Carrageenan | 0.60 |
| Sodium Bicarbonate | 0.50 |
| Bio-citrus | 1.00 |
| Green Tea Extract | 0.05 |
| Herbal Blend - Calendula, Cat's Claw, | 0.25 |
| Olive Leaf, Cleavers, Rhodeola Rosea | |
| Water | 57.3 |

### EXAMPLE 3

### Unscented Deodorant Gel

| Ingredient | % wt/wt |
|---|---|
| Glycerine | 30.00 |
| Sodium Stearate | 0.20 |
| Kappa Carrageenan | 0.30 |
| Iota Carrageenan | 0.60 |
| Lichen | 6.00 |
| Bio-citrus | 0.50 |
| Herbal Blend - Calendula, Cat's Claw, | 0.25 |
| Olive Leaf, Cleavers | |
| Water | 62.15 |

### EXAMPLE 4

### Hibiscus Deodorant Gel

| Ingredient | % wt/wt |
|---|---|
| Glycerine | 30.00 |
| Kappa Carrageenan | 0.30 |
| Iota Carrageenan | 0.30 |
| Hibiscus Extract | 0.50 |
| Bio-citrus | 1.00 |
| Herbal Blend - Calendula, Cat's Claw, | 0.25 |
| Olive Leaf, Cleavers | |
| Water | 67.65 |

### EXAMPLE 5

### Bamboo Deodorant Cream

| Ingredient | % wt/wt |
|---|---|
| Glycerine | 20.00 |
| Sodium Stearate | 1.50 |
| Kappa Carrageenan | 0.30 |
| Iota Carrageenan | 0.30 |
| Usnic Acid | 0.50 |
| Bamboo Extract | 0.50 |
| Herbal Blend - Calendula, Cat's Claw, | 0.25 |
| Olive Leaf, Cleavers | |
| Water | 76.65 |

### EXAMPLE 6

### Bois De Rose Deodorant Spray

| Ingredient | % wt/wt |
|---|---|
| Glycerine | 20.00 |
| Kappa Carrageenan | 0.10 |
| Bio-Citrus | 1.00 |
| Essential Oils | 0 |
| Rosewood | 0.50 |
| Geranium | 0.20 |
| Lemon | 0.30 |
| Herbal Blend - Calendula, Cat's Claw, | 0.25 |
| Olive Leaf, Cleavers | |
| Water | 77.65 |

### EXAMPLE 7

### Lavender and Beta Glucan Deodorant Stick

| Ingredient | % wt/wt |
|---|---|
| Glycerine | 40.00 |
| Iota Carrageenan | .8 |
| Bio-Citrus | 1.00 |
| Lavender | 0.50 |
| Sodium Stearate | 6.00 |
| 1,3/1,6 Beta Glucan | 3.00 |
| Herbal Blend - Rhodeola rosea, | 0.25 |
| Arabinogalactan | |
| Water | 48.45 |

We claim:

1. A deodorant composition suitable for application onto the skin, primarily free of ethoxylates or other petrochemical derivatives comprising:
(a) at least one of
(1) glycerine (preferably of plant origin);
(2) a polyol selected from the group consisting of galactitol, erythritol, inositol, ribitol, dithioerythritol, dithiothreitol;
(3) a sugar alcohol, selected from the group consisting of mannitol, sorbitol, xylitol and maltitol;
(4) a hydrogenated starch hydrosylates of at least one of berries, apples or plums, and
(5) mixtures thereof;
(b) water or a lower monohydric alcohol, selected from the group of methanol, ethanol, propanol and isoproponal, or mixtures thereof, present at a combined concentration of at least 20%;
(c) one or more carrageenans (preferably of plant origin) or alginates, or mixtures thereof, present in combined concentrations of less than 2%; and
(d) an effective amount of at least one natural deodorant ingredient;
(e) optionally, one or more thickeners or gums selected from the group consisting of tara, guar, xanthan, Arabic, tragacanth, agar, locust bean gum, ghatti and microcrystalline celluloses;
wherein the combined concentration of water, a lower monohydric alcohol and one or more of glycerine, galactitol, erythritol, inositol, ribitol, dithioerythritol, dithiothreitol, mannitol, sorbitol, xylitol, maltitol, or hydrogenated starch hydrolysates of berries, apples or plums, is at least about 60%.

2. A deodorant composition according to claim 1 further comprising one or more of
(a) sodium stearate, potassium stearate or calcium stearate, each at a concentration of from about 0.1% to about 10%;
(b) soapnut at a concentration of from about 0.001% to about 10%;
(c) rice bran extract from about 0.001% to about 0.5%; or
(d) Larch arabinogalactan at a concentration of from about 0.1% to about 5%.

3. A deodorant composition according to claim 1 wherein the natural deodorant ingredient is selected from the group consisting of (a) limonoid glucosides or bioflavonoids derived from the peel, seeds and pulp of citrus fruit, (b) lichens from the genus Usnea, (c) usnic acid and (d) mixtures thereof, at a concentration of from about 0.01% to about 10%.

4. A deodorant composition according to claim 1 further comprising one or more essential oils alone or in combination with one or more fragrances and/or hydrosols, at a combined concentration of from about 0.1% to about 20%.

5. A deodorant composition according to claim 4 wherein the essential oil is selected from the group consisting of rosewood, lavender, litsea cubeba, tea tree, lemon, lime, orange, petitgrain, geranium, lemongrass, palmarosa, mandarin, coriander, rose, yarrow, cypress, citronella, bergamot, pine and myrtle.

6. A method of suppressing body odor comprising applying to a body area where odor suppression is desired the deodorant composition of claim 1.

7. A method of suppressing body odor comprising applying to a body area where odor suppression is desired the deodorant composition of claim 2.

8. A method of suppressing body odor comprising applying to a body area where odor suppression is desired the deodorant composition of claim 3.

9. A method of suppressing body odor comprising applying to a body area where odor suppression is desired the deodorant composition of claim 4.

10. A deodorant composition according to claim 1, wherein the deodorant composition is applied from a pump, spray container, aerosol container, gel container, stick container, cream container, roll-on container or powder container.

11. A deodorant composition according to claim 1 that is substantially free of ethoxylates or other petrochemical derivatives.

12. The deodorant composition according to claim 1 that is essentially free of ethoxylates or other petrochemical derivatives.

13. A deodorant composition according to claim 1 that is free of ethoxylates or other petrochemical derivatives.

14. A topical delivery system primarily free of ethoxylates or other petrochemical derivatives comprising:
(a) at least one of
(1) glycerine (preferably of plant origin);
(2) a polyol selected from the group consisting of galactitol, erythritol, inositol, ribitol, dithioerythritol, dithiothreitol;
(3) a sugar alcohol, selected from the group consisting of mannitol, sorbitol, xylitol and maltitol;
(4) a hydrogenated starch hydrosylates of at least one of berries, apples or plums, and
(5) mixtures thereof,
(b) water or a lower monohydric alcohol, selected from the group of methanol, ethanol, propanol and isoproponal, or mixtures thereof, present at a combined concentration of at least 20%;
(c) one or more carrageenans (preferably of plant origin) or alginates, or mixtures thereof, present in combined concentrations of less than 2%; and
(d) sodium stearate at a concentration of from about 0.3% to about 10% or soapnut at a concentration of from about 0.1 to about 10%; and
(e) optionally, one or more thickeners or gums selected from the group consisting of tara, guar, xanthan, Arabic, tragacanth, agar, locust bean gum, ghatti and microcrystalline celluloses.

15. A topically-applied composition according to claim 14 further comprising a natural ingredient selected from the group consisting of (a) limonoid glucosides or bioflavonoids derived from the peel, seeds and pulp of citrus fruit, (b) lichens from the genus Usnea, (c) usnic acid and (d) mixtures thereof, at a concentration of from about 0.01% to about 10%.

16. A topically-applied composition according to claim 14 further comprising an active agent selected from the group consisting of antioxidants, free radical scavengers, vitamins, minerals and immuno-supportive ingredients.

17. A topically-applied composition according to claim 14 where the immuno-supportive ingredient is a β glucan present at a concentration of from about 0.1% to about 10%.

18. A topically-applied composition according to claim 17 where the β glucan is a 1,3 β glucan with 1,3/1,6 glucose linkages.

19. A deodorant composition of claim 1 suitable for application to household surfaces including floors, counter tops, sinks, rugs, upholstery, carpets, textiles, apparel, fur, faux fur and air.
